(19) United States
(12) Patent Application Publication
(10) Pub. No.: US 2026/0066112 A1
(43) Pub. Date: Mar. 5, 2026

(54) SYSTEMS AND METHODS FOR DENTAL DISEASE RISK PREDICTION AND DENTAL TREATMENT PLANNING USING MULTIMODAL ARTIFICIAL INTELLIGENCE

(71) Applicant: Shervin Molayem, Beverly Hills, CA (US)

(72) Inventor: Shervin Molayem, Beverly Hills, CA (US)

(21) Appl. No.: 18/822,388

(22) Filed: Sep. 2, 2024

(51) Int. Cl. G16H 50/20; G16H 20/40; G16H 50/30

(52) U.S. Cl. CPC G16H 50/20; G16H 20/40; G16H 50/30

(57) ABSTRACT

Systems and methods for a multimodal artificial intelligence processing to generate a unified perspective of a dental patient's diagnosis that includes dental disease risk prediction and treatment planning. According to an aspect, a multimodal artificial intelligence system can implement a computer-implemented method that includes acquiring dental data for a patient, processing the dental data for consumption by a machine learning model, configuring the machine learning model to be a treatment planning machine learning model, generating, via the treatment planning machine learning model, a patient treatment plan that includes at least one of a dental treatment plan and a predicted dental treatment plan, and displaying, via a graphical user interface (GUI), the generated patient treatment plan.

### TECHNICAL FIELD

The presently disclosed subject matter generally relates to dental treatment planning. Particularly, the presently disclosed subject matter generally relates to systems and methods for dental disease risk prediction and dental treatment planning using multimodal artificial intelligence.

### BACKGROUND

In recent years, advances in technology have led to significant growth in AI medical algorithms and tools making it possible for health professionals and practitioners to incorporate medical AI algorithms and tools into their specialties, procedures, and patient diagnosis thereby improving their accuracy, efficiency, and overall performance. To assist health professionals and practitioners, medical AI algorithms have been integrated into numerous existing tools, software, imaging, and measurements including, for example, AI implemented in diagnostic aids to automatically identify critical findings, AI automation of time-consuming functions such as quantification, contouring and auto complete of text in reports, AI workflow improvements and automation, and AI data mining applications. Other benefits of medical AI algorithms include modality-specific AI to iso-center patients, choose imaging protocols, or speed imaging exam time, AI to enhance image reconstruction, to improve image quality, resolution, and identify and fix imaging artifacts to help imagers get the best possible images, even if they are novice users of the system or are unfamiliar with the anatomy.

Augmenting AI medical algorithms and tools with a specialist's equipment, skill, and procedure can facilitate consistent decision-making, resulting in proper patient diagnosis and treatment. Further, specialists that implement vetted AI medical algorithms can help remove uncertainty in dental care giving patients peace of mind in following a necessary procedure or treatment plan prescribed by the specialists. Moreover, integrating several sets of medical AI algorithms and tools can provide specialists and the patient with a better understanding of the patient's health and health concerns.

As an example, in dentistry, patients may have complex care needs requiring coordination of multiple specialists to design and execute a particular treatment plan. Furthermore, treatment planning can be subjective, with different specialists exhibiting biases specific to their unique training often making it time consuming, expensive, and impractical for multiple specialists to collaborate to design individualized treatment plans for patients. Thus, in dentistry and other medical specialties, there is a need for artificial intelligence systems to assist in patient treatment planning.

### SUMMARY

The presently disclosed subject matter relates to systems and methods for dental disease risk prediction and dental treatment planning using multimodal artificial intelligence. According to an aspect, a computer-implemented method is described that includes acquiring a plurality of dental data for a patient, processing the plurality of dental data for consumption by a machine learning model, configuring the machine learning model to be a treatment planning machine learning model, generating, via the treatment planning machine learning model, a patient treatment plan that includes at least one of a dental treatment plan and a predicted dental treatment plan, and displaying, via a graphical user interface (GUI), the generated patient treatment plan. In embodiments, a computer-implemented method is described that includes acquiring a plurality of dental data for a patient, processing the plurality of dental data for consumption by a machine learning model, predicting, using a multilabel classification machine learning model, a patient's future risk of dental or systemic disease based on at least one of the processed plurality of dental data, and displaying, via a GUI, the multilabel classification machine learning model predictions of future disease risk, wherein the predicted future disease risk includes at least one of the following predicted diseases: periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and progression of cavities (incipient vs. in dentin, duration of time for cavity to progress into the nerve/pulp), diabetes, cancers, heart attacks/cardiovascular disease, stroke, inflammatory disease, and/or arthritis. In embodiments, a computer-implemented method is described that includes acquiring a plurality of dental data for a patient, processing the plurality of dental data for consumption by a machine learning model, predicting, using a multiclass classification machine learning model, a patient's treatment success based on at least one of the processed plurality of dental data, wherein an output classes of the multiclass classification machine learning model includes at least one of a treatment success, a treatment survival, and a treatment failure, and displaying, via a GUI, the multiclass classification machine learning model predictions.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent embodiments of the boundaries. In some embodiments one element may be implemented as multiple elements or that multiple elements may be implemented as one element. In some embodiments, an element shown as an internal component of another element may be implemented as an external component and vice versa. Furthermore, elements may not be drawn to scale.

FIG. 1 illustrates a block diagram of a computing system configured to provide dental disease risk prediction and dental treatment planning using multimodal artificial intelligence in accordance with embodiments of the present disclosure;

FIG. 2 illustrates a flow diagram of a method for dental or systemic disease risk prediction, predicting treatment success, and dental treatment planning using multimodal artificial intelligence processing;

FIG. 3 illustrates a flow diagram of a method for processing phases of a treatment plan option for an example treatment plan; and

FIG. 4 illustrates an embodiment of a computing system configured with the example systems and/or methods disclosed.

### DETAILED DESCRIPTION

Systems and methods are described herein as associated with a computer-implemented multimodal AI-driven system that offers dental disease risk prediction and treatment planning by constructing a unified perspective of a dental patient's diagnosis to address dental and systemic diseases, symptoms, and conditions. In certain embodiments, the unified perspective can include automatically synthesizing dental data including, for example, dental images, dental radiology reports, genetic testing results, physical examination findings, smoking status, demographics, color photographs, laboratory tests, existing diagnoses, dental procedure history, gum disease metrics, probing depth, or pain scores. The multimodal AI-driven system may integrate a wide array of digital and sensor-based diagnostics data to provide dental diagnosis and treatment planning.

In embodiments, a patient's diagnosis may be individualized by incorporating patient-specific data (or “patient data”) including, for example, a patient's personal goals for dental function vs. aesthetics, patient information and concerns, comfort, medical history, financial considerations, age, sex, and other factors. As an example, a patient may opt for one or more personal goals that include at least one of the following concepts: restoring or improving oral function, oral health, systemic health, improving aesthetics, maintaining low treatment cost, alleviating pain, and/or increasing comfort. Moreover, multimodal AI-driven system can provide dental treatment plans that include multiple alternative treatment plan options that may vary in complexity, cost, or likely outcome, and wherein each treatment plan option describes different treatments, interventions, or actions in chronological order.

In some examples, the multimodal AI-driven system may be integrated with a graphical user interface (GUI) designed specifically for dental professionals, allowing for easy input of patient data, interaction with the AI treatment planning process, and interpretation of the AI system's recommendations. Further, the multimodal AI-driven system GUI may display a generated treatment plan as well as displaying all of the inputs provided to a particular artificial intelligence model, for example, displaying of all of the information collected about a particular patient or a display of a patient's entire dental record. In certain embodiments, dental data can be interpreted, collected, processed, presented, and displayed to at least one of an interface coupled to a machine (e.g., robotic systems) or a computing device integrated with the multimodal AI-driven system.

In some examples, collected patient data and dental data may be pre-processed according to standard techniques used for preparing data for AI systems. For example, image data may be processed into a format suitable for AI analysis. Image processing may include normalization techniques, image data augmentation techniques, and image/pixel to array transformations. In some embodiments, patient and/or dental data having continuous variables may be centered and normalized using one or more statistical methods in the training dataset, as an example. In certain embodiments, categorical variables may be represented using one-hot or multi-hot encoding, and so forth. Moreover, the multimodal AI-driven system may implement a dental foundation model that may be trained to learn the data distribution of patient dental data. The data sources used for training a machine learning model may include at least one of patient examination data, as well as data obtained from the Internet, including dental research papers, publicly available genetic datasets, publicly available dental or medical imaging datasets, or articles related to dentistry, online resources, online databases, or collection of patient dental data from an electronic dental record. As an example, a machine learning model may be a generative model implemented as a neural network, for example using a Transformer architecture whereby the data may be represented as tokens and the training objective may be a next token prediction objective.

In embodiments, a multimodal AI-driven system may provide a treatment planning model to predict one or more dental treatment plans for a variety of different dental conditions including gingivitis, cavities, dentin hypersensitivity, halitosis, impacted teeth, chipped teeth, broken teeth, crooked teeth, stained teeth, tooth pain, malocclusion, tooth erosion, mouth ulcers, bruxism, and/or temporomandibular joint disorders. In certain embodiments, a treatment plan may include multiple alternative treatment plan options, which may vary in complexity, cost, or likely outcome. Each treatment plan option may describe different treatments, interventions, or actions in chronological order. Moreover, each treatment plan option may include one or more phases and/or procedures. Phases may include descriptions of specific interventions on particular teeth. A treatment plan option may be comprehensive, or a “band aid” approach solving only one specific problem.

In some examples, the treatment planning model may be used during a procedure. A dentist or other care team member may take a photograph of a patient during a procedure and provide this photograph as input to the treatment planning model in order to receive feedback about the next steps needed in the patient's care. In such cases, the multimodal AI-driven system may provide an AI treatment planning model to help resolve unexpected issues that arise during a procedure.

In embodiments, the multimodal AI-driven system may leverage a neural network architecture having one or more neural network layers. In some embodiments, the multimodal AI-driven system may include a feedforward neural network to process non-image data, while a convolutional neural network (CNN) or vision transformer architecture (VTA) may be used to process the image data. A transformer architecture may be used to process image data and/or to process any data represented as tokens. A unified architecture may be developed in which the following are concatenated: one or more representations produced by one or more feedforward neural networks, one or more representations produced by one or more convolutional neural networks, and/or one or more representations produced by one or more Transformers. One or more additional neural network layers may then be applied to the concatenated representation in order to produce the final model output.

In embodiments, a multimodal AI-driven system may predict a patient's future risk of oral or systemic disease based on dental health information and/or general health information by applying a multilabel classification model, as an example. The multilabel classification model may also forecast long-term oral health outcomes based on current data. In certain embodiments, the output of the model can include dental diseases, conditions, or symptoms that may be predicted including periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and/or progression of cavities (incipient vs. in dentin, duration of time for cavity to progress into the nerve/pulp). In some embodiments, the output of the model can include systemic diseases, conditions, or symptoms that may be predicted, for example, diabetes, cancers, heart attacks/cardiovascular disease, stroke, inflammatory disease, and arthritis. Additional tongue conditions may be predicted using one or more color photographs or other measurements of the tongue, including median rhomboid glossitis, atrophic glossitis, fissured tongue, geographic tongue, hairy tongue, oral hairy leukoplakia, lichen planus, linea alba, leukoplakia, squamous cell carcinoma, papilloma, tongue tie, and macroglossia. Systemic conditions may also be predicted based on tongue images or other tongue data, including vitamin and mineral deficiencies causing a change in tongue color, and/or infections causing changes in appearance (e.g. “strawberry tongue” seen in scarlet fever, or syphilitic glossitis seen in tertiary syphilis).

In certain embodiments, the multimodal AI-driven system may include a treatment success model to predict treatment success by comparing pre-treatment patient data with post-treatment patient data, or by analyzing post-treatment patient data alone. The multimodal AI-driven system may implement a comparison model having model inputs that may include any or all of the patient data and/or dental data at a pre-treatment time point, as well as any or all of the patient data and/or dental data at a post-treatment time point. In a post-treatment data model, only the post-treatment time point data will be included as input. As an example, the model may be implemented as a multiclass classification model with three outputs: treatment success, treatment survival, or treatment failure. Further, the model may also be implemented using a regression objective to predict a numerical score indicative of the degree of treatment success. The dataset for training and evaluation may be collected from electronic health record software. For a comparison model, pre-treatment and post-treatment data can be obtained from, for example, x-rays before treatment along with x-rays after treatment.

Previous dental treatment planning methods and systems for providing patient care relied heavily on the judgment and expertise of dental practitioners. This approach can lead to variations in diagnoses and treatment plans, potentially affecting the quality and consistency of patient care. Furthermore, professionals from different subspecialties may offer divergent perspectives on the ideal treatment plan for a particular patient. Further, current technologies in dental treatment planning include standalone diagnostic tools and manual integration of patient data by practitioners. These methods are limited to reliance on subjective interpretation and the disjointed use of diagnostic information leading to potential inconsistencies in treatment planning.

With the present multimodal AI-driven system, an AI-driven system may be configured with a number of AI/ML models that incorporate diverse data sources, including dental images, sensor data, gum disease metrics, genetic test results, physical examination findings, patient history, and patient data to construct a unified perspective of a dental patient's diagnosis. The multimodal AI-driven system models may be used to facilitate dental disease risk prediction, treatment planning, and individualized treatment plans based on patient data and dental data. Moreover, the use of AI/ML models within the multimodal AI-driven system may reduce reliance on subjective judgment and has the potential to improve diagnostic accuracy and treatment outcomes. The AI system may be especially useful for complex cases where multidisciplinary expertise is needed. Also, the AI system can facilitate multidisciplinary cases where the information of all the dental specialties are inputted and accounted for in the planning of large complex cases. Further, the multimodal AI-driven system may be configured to implement predictive analytics via disease progression modeling to determine treatment progress, success, deviations, or issues and forecast disease progression thereby allowing dental professionals to intervene early in disease progression to prevent or mitigate worsening dental conditions. The multimodal AI-driven system can provide patients with a personalized treatment plan leading to more accurate, efficient, and patient-centric care.

With the present multimodal AI-driven system, an AI-driven system may be routinely and periodically implemented to assess a patient's treatment success allowing dental professionals to routinely monitor and identify whether a treatment plan or procedure is achieving its intended goals. In cases where the AI system detects deviations or potential issues, recommendations and suggested adjustments to the treatment plan or alternative approaches can be offered. This real-time feedback and support can be invaluable in maintaining treatment efficacy and ensuring patients receive the best care. Through routine consults and checkups, the multimodal AI-driven system can enable early detection and intervention thereby enhancing the overall oral health outcomes for patients. These and other features are described herein with reference to the attached figures.

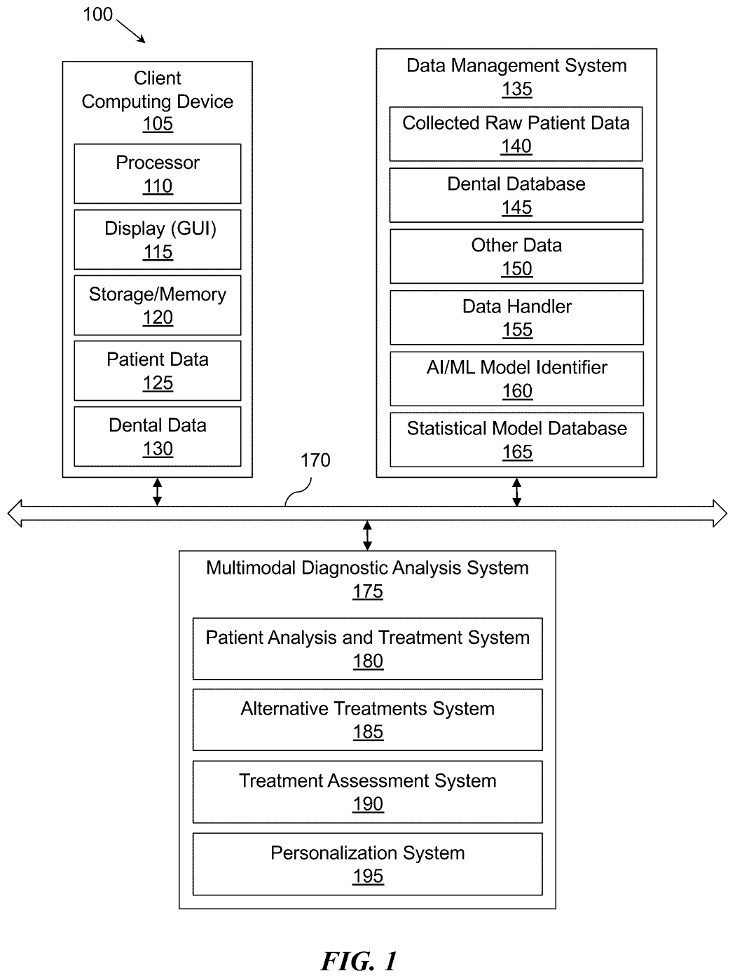

With reference to FIG. 1, the figure illustrates a block diagram of a computing environment configured with a multimodal AI system 100. In embodiments, the multimodal AI system 100 (hereinafter “MAI system 100”) is configured to autonomously execute with and/or be integrated with a selected software application that includes a data management system 135 and a multimodal diagnostic analysis system 175. In embodiment, the MAI system 100 may be configured to provide dental disease risk prediction and dental treatment planning using multimodal artificial intelligence. In embodiments, the multimodal artificial intelligence system can be configured to incorporate diverse data sources, including dental images, dental radiology reports, smoking status, demographics, color photographs, laboratory tests, existing diagnoses, dental procedure history, probing depth, pain scores, and so forth. sensor data, gum disease metrics, genetic test results, physical examination findings, patient history, and patient data to construct a unified perspective of a dental patient's diagnosis. In some embodiments, the data management system 135 and/or multimodal diagnostic analysis system 175 may include, but is not limited to, a computer application/program that includes one or more algorithms configured to generate one or more results based on one or more input values. The algorithm comprises a set of generative, neural network, multiclass classification, multilabel classification, generative pre-trained Transformer, dental foundation model and/or functions that generate a result based on data. Further, the data management system 135 and/or multimodal diagnostic analysis system 175 may be implemented within a computing device, a machine (e.g., a robotic device), a server operating in a cloud computing system, a server configured in a Software as a Service (SaaS) architecture, a smart phone, laptop, tablet computing device, and so on.

Further, the multimodal AI-driven system may be configured with a number of AI models that provide dental disease risk prediction, treatment planning, and individualized treatment plans based on patient data and dental data. The use of AI/ML models within the multimodal AI-driven system can reduce reliance on subjective judgment and has the potential to improve diagnostic accuracy and treatment outcomes.

In embodiments, the MAI system 100 is configured to predict a patient's future risk of oral or systemic disease based on dental health information and/or general health information by applying a multilabel classification model, as an example. In certain embodiments, the MAI system 100 is configured with a treatment success model to predict treatment success by comparing pre-treatment patient data with post-treatment patient data, or by analyzing post-treatment patient data alone. In some embodiments, the MAI system 100 is configured with model inputs that may include any or all of the patient data and/or dental data at a pre-treatment time point, a post-treatment time point, and so forth.

As an example, at least one of a treatment planning, disease risk prediction, and treatment assessment AI model(s) of the MAI system 100 may be implemented using a multiclass classification model with three outputs: treatment success, treatment survival, or treatment failure. The AI model may be configured to further implement a regression objective to predict a numerical score indicative of the degree of treatment success, disease risk, and so forth. In embodiments, the data management system 135, and/or multimodal diagnostic analysis system 175 may be implemented as a set of computer applications/programs, functions, or algorithms configured to generate one or more results based on one or more input values. In embodiments, the MAI system 100 may include, but is not limited to, a computer application/program that includes one or more algorithms configured to generate one or more results based on one or more input values. The algorithm may comprise of a set of generative, neural network, multiclass classification, multilabel classification, generative pre-trained Transformer, dental foundation model and/or functions that generate a result based on data.

As shown in FIG. 1, the computing environment (e.g., a cloud-computing environment) of the MAI system 100 may provide access to remote client devices such as client computing device 105 through one or more network communication channels 170 (e.g., a communication bus, wireless communication, wired networks, combinations of channels, etc.). The client device may include one or more processors 110, a display that includes a graphical user interface (GUI) 115 for displaying patient data 125 and/or dental data, as well as a retrieving a dental patient's diagnoses, dental disease risk predictions, patient's future risk of oral or systemic disease predictions, one or more dental treatment plans or procedures, an individualized patient treatment plan, and an assessment of the success of a treatment and/or procedure as obtained from the data management system 135 and multimodal diagnostic analysis system 175, as described herein. The client device may access and communicate patient data 125 and/or dental data 130 via a graphical user interface 115 to run, for example, AI/ML (e.g., generative) models and functions through the MAI system 100. The MAI system 100 is configured to analyze the contents of the patient data 125 and/or dental data 130, screen the patient data 125, if needed, to remove sensitive information, and determine models for processing the patient data 125 and/or dental data 130 to generate a unified perspective of a dental patient's diagnosis to provide at least one of dental disease risk predictions, prediction of a patient's future risk of oral or systemic disease, a dental treatment plan, an individualized patient treatment plan, and an assessment of the success of a treatment and/or procedure. In embodiments, the MAI system 100 may aggregate and display patient and dental data, executed models, functions, alerts, and other details and information in a map, chart, diagram, or tabular form on a graphical user interface (GUI).

With reference to FIG. 1, embodiments of a computing environment is illustrated that is configured with a multimodal AI system 100 for obtaining and processing a patient's data. As an example, the patient data 125 may include a patient's health or medical history, a patient's personal goals for dental function vs. aesthetics, other patient information, comfort, conditions, and concerns, insurance information, finances, financial considerations, age, sex, and other factors. Moreover, patient data 125 may include one or more personal goals that include at least one of the following concepts: restoring or improving oral function, oral health, systemic health, improving aesthetics, maintaining low treatment cost, alleviating pain, and/or increasing comfort. As an example, the patient's personal treatment goals may be represented in natural language, vector representations of words, a one-hot vector, or a multi-hot vector. The patient data 125 may be used by the MAI system 100 to generate a patient data report, the patient data 125 may be stored in storage/memory 120 and communicated to data management system 135 and/or multimodal diagnostic analysis system 175 for further processing. In embodiments, the MAI system 100 may provide a guided patient workflow, via a graphical user interface (GUI), that includes, for example, a sequence or series of selections and questions for creating, configuring, or updating to complete a clinical profile of a patient and generate patient data 125. The patient data 125 may be provided by the client computing device 105 directly to the MAI system 100 whereby the patient data may be processed by the multimodal diagnostic analysis system 175, stored on the client device storage 120, stored on multimodal diagnostic analysis storage 190, and processed by data management system 135, or any combination thereof.

With reference again to FIG. 1, the computing environment illustrated may be configured with a multimodal AI system 100 for obtaining and processing a dental data 130 that may include dental data acquired directly from the patient and dental data from various data sources relevant to the patient's health, symptoms, conditions, diagnosis, and the like. As an example, the dental data 130 may include patient's health information collected from diverse data sources, including dental images, dental radiology reports, smoking status, demographics, color photographs, laboratory tests, existing diagnoses, dental procedure history, probing depth, pain scores, sensor data, gum disease metrics, genetic test results, physical examination findings, patient history, and patient data to construct a unified perspective of a dental patient's diagnosis. In certain embodiments, the dental data can include the patient's personal treatment goals represented in natural language, vector representations of words, a one-hot vector, or a multi-hot vector, and wherein the personal treatment goals include at least one of the following concepts: restoring or improving oral function, oral health, systemic health, improving aesthetics, maintaining low treatment cost, alleviating pain, and/or increasing comfort. The dental data 130 may be used by the MAI system 100 to generate a patient data report, the dental data 130 may be stored in storage/memory 120 and communicated to data management system 135 and/or multimodal diagnostic analysis system 175 for further processing. In embodiments, the MAI system 100 may provide a guided patient workflow, via a graphical user interface (GUI), that includes, for example, a sequence or series of selections and questions for creating, configuring, or updating to complete a clinical profile of a patient and generate dental data 130. The dental data 130 may be provided by the client computing device 105 directly to the MAI system 100 whereby the dental data may be processed by the multimodal diagnostic analysis system 175, stored on the client device storage 120, stored on multimodal diagnostic analysis storage 190, and processed by data management system 135, or any combination thereof.

The client device 105 may include a processor 110 having any number of microprocessors, microcontrollers, graphics processing units (GPUs), central processing units (CPUs), quantum processing units (QPUs), artificial intelligence processing units (AI PUs), neural processing units (NPUs), tensor processing units (TPUs), analog circuitry, or the like that may be programmed to execute computer-executable instructions for implementing aspects of this disclosure.

The client device 105 may include a graphical user interface (GUI) display 115 that may be integrated with the MAI system 100 and designed for dental professionals, allowing for easy input of patient data 125, dental data 130, as well as easy interaction with the AI treatment planning process, and interpretation of the AI system's recommendations. The GUI display 115 may include any of the following elements: an interface for uploading files that are elements of patient dental records, such as uploading results of laboratory tests or uploading DICOM image files derived from dental imaging, an interface for manually entering text data or numerical data corresponding to elements of patient dental records, a display of all of the inputs provided to a particular artificial intelligence model, such as display of all of the information collected about a particular patient or a display of a patient's entire dental record, a display of the generated treatment plan.

The client device 105 may include a storage/memory 120 configured with at least one of a semiconductor memory, a magnetic disk device, and/or an optical disk device, or the like, for example. The storage/memory 120 may also store and run application, programs, algorithms, functions, or software for accessing the multimodal diagnostic analysis system 175 and/or data management system 135, as well as storing and accessing textual information, visual information, and other content related to patient data and dental data of one or more patients. The storage/memory 120 may store executable code of a graphical user interface (GUI) to allow a user to display, configure, edit, and navigate through the multimodal diagnostic analysis system 175 and/or data management system 135.

In some embodiments, the example network communication channel(s) 170 may be a network environment, for example, a distributed client/server system that spans one or more networks. The network communication channels 170 may be a small computer network such as a local CDN network, P2P network, local area network (LAN), edge computing network, or local or hyperlocal distributed computing network. In some embodiments, the network communication channels 170 may be a large computer network such as, for example, a wide area network (WAN), the Internet, a cellular network, or a combination thereof connecting any number of mobile clients, fixed clients, and servers. In embodiments, the network communication channels 170 allows software, code, instructions, and data to be transferred between the client computing device 105, the multimodal diagnostic analysis system 175, and the data management system 135, and other external systems, databases, and devices communicably coupled thereto.

As described above, the MAI system 100 is configured to include a data management system 135 and a multimodal diagnostic analysis system 175. In embodiments, the data management system 135 may retrieve raw data and additional information associated with the raw data to determine a statistical model and one or more machine learning models for processing the raw data. In embodiments, the data management system 135 may obtain and process the raw data to remove sensitive or confidential information before any further processing is done using the raw data. In some embodiments, the data management system 135 may retrieve pre-processed raw data (screened raw data) having been screened for and removed of sensitive or confidential information. The raw data may be pre-processed by a user of client computing device and/or MAI system 100 then communicated to the multimodal diagnostic analysis system 175 to generate a unified perspective of a dental patient's diagnosis to facilitate dental disease risk predictions, prediction of a patient's future risk of oral or systemic disease, a dental treatment plan, an individualized patient treatment plan, and an assessment of the success of a treatment and/or procedure.

In embodiments, the data management system 135 may retrieve from a client computing device 105 (e.g., a source such as a storage system, computing system, or network environment) raw patient data 140 that may include visual content, textual information, natural language, vector representations of words, a one-hot vector, a multi-hot vector, meta data, or any combination thereof pertaining to a patient data, dental data, dental and systemic diseases, conditions, or symptoms for a patient, digital and/or sensor data, financial and insurance information, sensitive or confidential data, and other data 150 associated with the patient. Moreover, raw patient data 140 may include feature data, formulas, algorithms, and other data 150 associated with the client computing device 105, patient data 125, and dental data 130. In some embodiments, the raw patient data 140 may be processed by a data handler 155 to remove sensitive or confidential information, reconstruct or process the raw patient data 140 to obtain usable content for processing by the multimodal diagnostic analysis system 175. The data handler 155 may include one or more privacy preserving AI/ML models to identify, anonymize, or replace entries or data having sensitive, confidential, or personally identifiable information. In some embodiments, data handler 155 may aggregate, separate, sort, filter, or organize data into a tabular or usable format or layout for processing by artificial intelligence/machine learning (AI/ML) model identifier 160, statistical model databases 165, or the multimodal diagnostic analysis system 175. In certain embodiments, data handler 155 may determine features, feature vectors, content, and composition of datasets from patient data 125, dental data 130, collected raw patient data 140, or other data 150.

In embodiments, the data management system 135 may process the raw patient data 140 to remove sensitive or confidential information before any further processing is done using the raw patient data 140. In some embodiments, the data management system 135 may retrieve pre-processed raw patient data 140 (screened/processed data) having been screened for and removed of sensitive or confidential information. The raw data may be pre-processed by the multimodal diagnostic analysis system 175 or client computing device 105 then communicated to the data management system 135 to facilitate privacy.

The data management system 135 may retrieve from a dental database 145 dental data relevant to the patient's health used for training a machine learning model. The dental database 145 that may include data obtained from the Internet, including dental research papers, publicly available genetic datasets, publicly available dental or medical imaging datasets, or articles related to dentistry, online resources, online databases, or collection of patient dental data from an electronic dental record. The relevant dental data may be acquired from various data sources based on a patient's symptoms, conditions, diagnosis, and the like and may be used for processing by one or more AI/ML models as determined by the AI/ML model identifier 160 based on the content of a patient's data 125, dental data 130, raw patient data 140, or any combination thereof. In some embodiments, the dental database 145 may be stored remotely, in part or in whole, and accessed by the client computing device 105 or the MAI system 100 through one or more network communication channels 170 (e.g., a communication bus, wireless communication, wired networks, combinations of channels, etc.). As an example, the dental database 145 may include a repository or collection of diverse data sources, including dental data for dental diseases, symptoms, or conditions such as, for example, gingivitis, cavities, dentin hypersensitivity, halitosis, impacted teeth, chipped teeth, broken teeth, crooked teeth, stained teeth, tooth pain, malocclusion, tooth erosion, mouth ulcers, bruxism, and/or temporomandibular joint disorders. Further, the dental database 145 may include a repository or collection of diverse data sources for systemic diseases, conditions, or symptoms such as, for example, periodontal disease, tongue diseases, diseases that manifest through abnormal tongue appearance such as vitamin deficiency, bone loss, amount of tooth mobility, bone breakdown, diabetes, cancers, heart attacks/cardiovascular disease, and so forth.

In some embodiments, the relevant dental data from the dental database 145 may be processed by a data handler 155 to reconstruct or process the dental data to obtain usable content for processing by the multimodal diagnostic analysis system 175. In some embodiments, data handler 155 may aggregate, separate, sort, filter, or organize dental data into a tabular or usable format or layout for processing by AI/ML model identifier 160, statistical model databases 165, or the multimodal diagnostic analysis system 175. In certain embodiments, data handler 155 may determine features, feature vectors, content, and composition of datasets from dental database 145.

In some embodiments, data management system 135 may retrieve other data 150, including feature data, statistical data, data source, and data type associated with the raw patient data 140 and client computing device 105. The data management system 135 may include a statistical model database 165 that may be used for determining one or more predictive, generative, neural network, multiclass classification, multilabel classification, generative pre-trained Transformer, dental foundation model, or other AI/ML models for processing the collected raw patient data 140, relevant dental data from the dental database 145, or other data.

In certain embodiments, the dataset for training and evaluation may be collected from electronic dental record software and stored in dental database 145 and/or other data 150. The dataset for training and evaluation by machine learning models may be collected from electronic dental record software. As an example, a dental foundation model may be trained to learn the data distribution of patient dental data. The data used for training may be any or all of the data sources shown in Table 1 below, as well as data scraped from the Internet, including dental research papers, publicly available genetic datasets, publicly available dental or medical imaging datasets, or articles related to dentistry. In certain embodiments, the data may be represented as tokens and the training objective may be a next token prediction objective.

In embodiments, the data handler 155 may be configured to process the patient data 125, dental data 130, raw patient data 140, or relevant dental data from the dental database 145, other data 150, or any combination thereof, to determine a statistical model. The statistical model may be communicated to the AI/ML model identifier 160 to determine one or more predictive, generative, neural network, multiclass classification, multilabel classification, generative pre-trained Transformer, dental foundation model, or other AI/ML models for processing the patient data and dental data by the multimodal diagnostic analysis system 175. In some embodiments, the data management system 135 may retrieve feature data from raw patient data 140 and relevant dental data from the dental database 145 that may be used by AI/ML model identifier 160 to determine one or more predictive, generative, neural network, multiclass classification, multilabel classification, generative pre-trained Transformer, dental foundation model, or other AI/ML models.

The AI/ML model identifier 160 may include logic, memory, and processor for evaluating patient data 125, dental data 130, raw patient data 140, or relevant dental data from the dental database 145, other data 150, and statistical models from statistical model database 165 to determine one or more predictive, generative, neural network, multiclass classification, multilabel classification, generative pre-trained Transformer, dental foundation model, or other AI/ML models. In some embodiments, the data management system 135 may be a server, for example, a database server for storing and maintaining AI/ML models.

With reference to FIG. 1, embodiments of a multimodal AI-driven system environment is illustrated that is configured with a multimodal AI system 100. In embodiments, the multimodal AI system 100 is configured to include a multimodal diagnostic analysis system 175 which may predict a patient's future risk of dental or systemic disease, treatment and/or procedure success, and need for referral to a see a specialist based on patient data, dental data, and other data processed by data management system 135. The multimodal diagnostic analysis system 175 may then provide a listing of different treatments, interventions, or actions in chronological order. The multimodal diagnostic analysis system 175 may further provide a dental treatment plan that includes multiple alternative plan options based on complexity, cost, or likely outcome. The multimodal diagnostic analysis system 175 may facilitate a patient's personal treatment goal(s) by constructing a patient treatment plan where treatment and procedure options are classified by priority or ranking based on patient data. The multimodal diagnostic analysis system 175 may assess the success of a generated treatment by acquiring a post-treatment patient data, and a variety of dental treatment plans and treatment options.

The multimodal diagnostic analysis system 175, the MAI system 100, and/or client computing device 105 may be implemented as a robotics system for carrying out one or more treatments through direct physical intervention upon the patient. In some embodiments, the robotics system may be integrated with one or more machine learning models of the multimodal diagnostic analysis system 175 including, for example, predictive, generative, neural network, multiclass classification, multilabel classification, generative pre-trained Transformer, dental foundation model, or other AI/ML models. Further, the robotics system may carry out one or more treatment plans determined from the MAI system 100. As an example, the MAI system 100 may include, but is not limited to, a computer application/program having one or more algorithms configured to generate input commands to a robotics system that will then carry out the one or more proposed treatments. The data management system 135 may communicate collected raw patient data 140, relevant dental data from dental database 145, other data 150, one or more AI/ML models and statistical data as determined by AI/ML model identifier 160 and statistical model database 165 to the multimodal diagnostic analysis system 175.

The multimodal diagnostic analysis system 175 may include patient analysis and treatment system 180, alternative treatment system 185, treatment assessment system 190, and personalization system 195. In embodiments, any one of the patient analysis and treatment system 180, the alternative treatment system 185, the treatment assessment system 190, and the personalization system 195 may include, or be implemented as, a set of computer applications/programs, functions, or algorithms configured to generate one or more results based on one or more input values. In embodiments, the algorithms, functions, or applications/programs may comprise of a set of generative, neural network, multiclass classification, multilabel classification, generative pre-trained Transformer, dental foundation model and/or functions that generate a dataset result based on raw data communicated from the client computing device 105, processed data communicated from the data management system 135, or any combination thereof. As an example, at least one of the patient analysis and treatment system 180, the alternative treatment system 185, the treatment assessment system 190, and the personalization system 195 receives a plurality of dental data including at least one of: dental images, dental radiology reports, genetic testing results, physical examination findings, smoking status, demographics, color photographs, laboratory tests, existing diagnoses, dental procedure history, gum disease metrics, probing depth, or pain scores, and so forth.

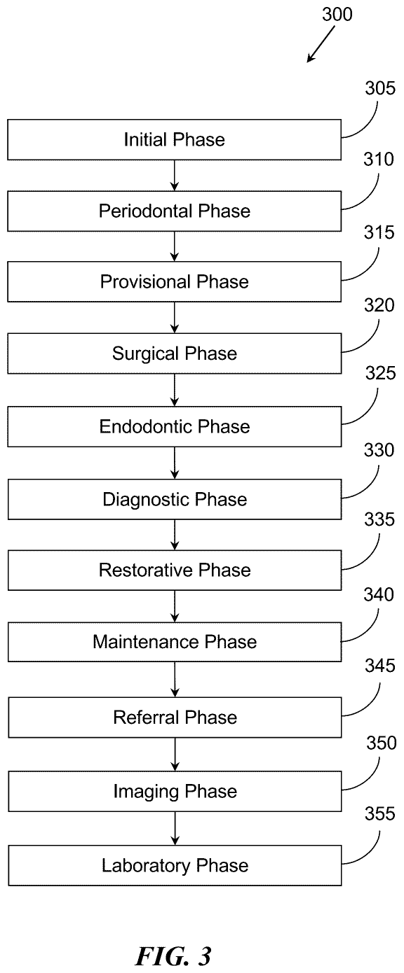

The patient analysis and treatment system 180 may implement one or more machine learning models as described herein to process data (i.e., screened/processed data) during a treatment or dental procedure in order to provide feedback about the next steps needed in the patient's immediate care. For example, the patient analysis and treatment system 180 may provide feedback during at least one dental treatment plan phase (shown in FIG. 3), including the initial phase, periodontal phase, provisional phase, surgical phase, endodontic phase, diagnostic phase, restorative phase, maintenance phase, referral phase, imaging phase, or laboratory phase.

In embodiments, the patient analysis and treatment system 180 may access and implement one or more machine learning models as described herein trained on an online resource, online database, or collection of patient dental data from an electronic dental record. The patient analysis and treatment system 180 may then implement a plurality of machine learning models to facilitate a unified treatment plan for an individual patient that addresses one or more of the following dental conditions: gingivitis, cavities, dentin hypersensitivity, halitosis, impacted teeth, chipped teeth, broken teeth, crooked teeth, stained teeth, tooth pain, malocclusion, tooth erosion, mouth ulcers, bruxism, or temporomandibular joint disorders.

In certain embodiments, the patient analysis and treatment system 180 may obtain and process the patient's processed data dental data to predict a patient's treatment success. The patient analysis and treatment system 180 may utilize a multiclass classification machine learning model to process at least one of the patient data or processed plurality of dental data to predict a patient's treatment success. The output classes of the multiclass classification machine learning model may include treatment success, treatment survival, or treatment failure. The results and model predictions may be displayed via the display GUI 115.

In some embodiments, the patient analysis and treatment system 180 may obtain and process the patient's raw data and/or processed dental data to predict a patient's future risk of dental or systemic disease. The patient analysis and treatment system 180 may utilize a multilabel classification machine learning model to process at least one of the raw data or processed plurality of dental data to predict a patient's future risk of dental or systemic disease. The output classes of the multiclass classification machine learning model may include treatment success, treatment survival, or treatment failure. The results and model predictions may be displayed via the display GUI 115. For example, patient risk of dental or systemic diseases that may be predicted include periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and/or progression of cavities (incipient vs. in dentin, duration of time for cavity to progress into the nerve/pulp), diabetes, cancers, heart attacks/cardiovascular disease, stroke, inflammatory disease, and/or arthritis.

The patient analysis and treatment system 180 may obtain and process the patient's raw data and/or processed dental data to further predict the dental and gum health of the patient and the progression of existing dental disease using, for example, the multilabel classification machine learning model. Moreover, the patient analysis and treatment system 180 may utilize one or more machine learning models as described herein to predict a priority level for one or more elements of the generated treatment plan. The patient analysis and treatment system 180 may obtain and process the patient's raw data and/or processed dental data using one or more machine learning models as described herein to further predict whether or not a patient requires referral to a specialist, and if so, what specialist the patient should see.

The alternative treatment system 185 may obtain and process the patient data 125, dental data 130, raw patient data 140, or relevant dental data from the dental database 145 using one or more machine learning models as described herein to provide a dental treatment plan that includes multiple alternative treatment plan options based on complexity, cost, or likely outcome of a procedure, each treatment plan option may describe different treatments, interventions, or actions in chronological order.

The treatment assessment system 190 may obtain and process the patient's raw data and/or processed dental data using one or more machine learning models described herein to predict treatment success by comparing a pre-treatment patient data with a post-treatment patient data, or by analyzing a post-treatment patient data alone. In embodiments, the treatment assessment system 190 may utilized a multiclass classification machine learning model to predict a patient's treatment success based on at least one of a processed plurality of dental data, or raw data. The multiclass classification machine learning model may be configured to include output classes such as a treatment success, a treatment survival, or a treatment failure. Moreover, the treatment assessment system 190 may assess the success of the generated treatment by acquiring a post-treatment plurality of patient data, providing the post-treatment patient data to a progression machine learning model along with the pre-treatment patient data, and receiving a model output indicating treatment success. The results and model predictions may be displayed via the display GUI 115.

The personalization system 195 may obtain and process as inputs, the patient's preferences as processed patient data, that may include, the patient's personal treatment goals represented in natural language, vector representations of words, a one-hot vector, or a multi-hot vector. The personalization system 195 may obtain and process the patient data 125, dental data 130, raw patient data 140, or relevant dental data from the dental database 145 using one or more machine learning models as described herein to provide a treatment plan that incorporates a patient's personal treatment goals that may include at least one of the following concepts: restoring or improving oral function, oral health, or systemic health, improving aesthetics, maintaining low treatment cost, alleviating pain, and/or increasing comfort.

### Multimodal AI Analysis and Data Processing Embodiments

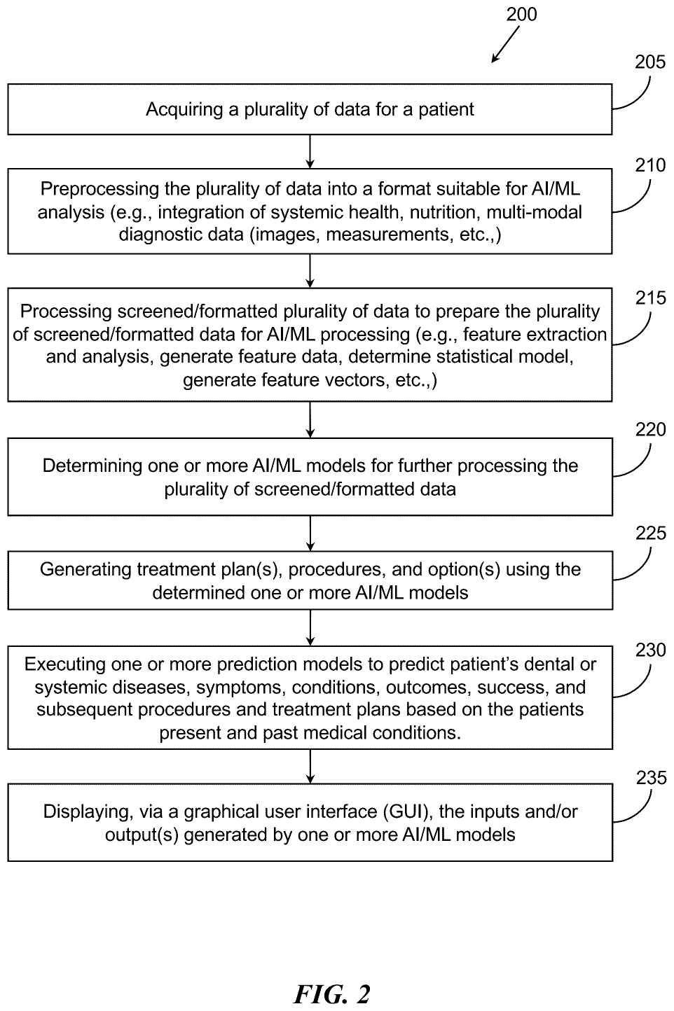

FIG. 2 illustrates a flow diagram of a method performed for dental disease risk prediction and treatment planning by preparing raw data for use in a multimodal artificial intelligence system, whereby the system processes a plurality of patient data to generate a unified perspective of a dental patient's diagnosis. In example provided herein, the method is described as being implemented by the system shown in FIG. 1, although it should be understood that the method may be implemented by any suitable system or computing device.

With continuing reference to FIG. 2, a unified perspective of a dental patient's diagnosis can include, for example, predicting treatment success based on the patient's dental data, predicting a patient's future risk of dental or systemic disease, predicting whether or not a patient requires referral to a specialist, facilitating a patient's personal treatment goal(s), providing a dental treatment plan that includes multiple alternative treatment plan options based on complexity, cost, or likely outcome of a procedure, assessing the success of a generated treatment by acquiring a post-treatment patient data, as well as a variety of dental treatment plans and treatment options. Blocks shown in FIG. 2 may represent one or more processes, methods or subroutines, carried out in the exemplary method. FIG. 1 and FIGS. 3 and 4 depict example embodiments of implementing the method of FIG. 2 for acquiring, preparing, and processing a plurality of patient data using a multimodal artificial intelligence system to generate a unified perspective of a dental patient's diagnosis that includes dental disease risk prediction and treatment planning.

Method 200 may be used independently or in combination with other methods or processes for creating a unified perspective of a dental patient's diagnosis that includes dental disease risk prediction and treatment planning. The method may include various programs, algorithms, logic, applications, systems, and AI/ML frameworks and models for evaluating screened raw data to determine suitable processes for generating a unified perspective of a dental patient's diagnosis. Each block shown in FIG. 2 may represent one or more processes, methods, or subroutines, carried out in the exemplary method. For explanatory purposes, the example process 200 is described herein with reference to FIG. 1 and FIGS. 3-4. Method 200 may be performed by the data management system 135, multimodal diagnostic analysis system 175, or both.

In embodiments, the method 200 may be implemented and performed by the MAI system 100 of FIG. 1. With the MAI system 100, a plurality patient data may be selected and entered into a plurality of machine learning models that when executed provide patients and dental professionals and practitioners with a unified perspective of a dental patient's diagnosis. The executed models and patient data may be stored in a database and made available for access to patients and dental professionals and practitioners. Moreover, the stored executed models and patient data may be updated and processed at a later time with new information to provide patients with updated treatment options, predictions, referrals, alternative treatment options, and assessments of the success of a previously generated and/or undergone treatment, or any combination thereof. A non-exhaustive list of examples of a machine learning model includes generative, neural network, multiclass classification, multilabel classification, generative pre-trained Transformer, dental foundation, generative adversarial networks (GANs), generative pre-trained transformers (GPTs), and other generative models.

Method 200 of FIG. 2 begins at block 205, for example, when a user access the MAI system 100 and/or client computing system 105 and provides raw patient data and/or dental data that is acquired and processed by the data management system 135, the multimodal diagnostic analysis system 175, or both.

In block 205, the MAI system 100 and/or client computing system 105 may display a graphical user interface (GUI) configured for acquiring a plurality of data for a patient, as well as acquiring changes and updates to the plurality of data, settings, preferences, and other background information for updating or maintaining the MAI system 100, machine learning models, data handlers, dental database, and so forth. Input data to the MAI system 100 may include a plurality of data for a dental patient that describes a patient's dental and systemic health, symptoms, conditions, or diseases, as well provides or updates patient data and dental data for a dental patient as described herein. The proposed machine learning models may take as input any or all of data types listed in the non-exhaustive list of data sources described below in Table 1. The user provided input (e.g., patient data, dental data, etc.,) may be raw data and/or processed data that may need to be formatted or screened for determining an appropriate machine learning model for handling the input.

TABLE 1
Patient data sources related to dental and systemic health

| | | Input format to |
|---|---|---|
| | | model (before |
| Data source | Description | preprocessing) |
| Full mouth | X-rays of the entire mouth including | Pixel |
| x-rays | the teeth, gums, and jaw bones. | values |
| (FMX) | A series of 18-20 x-rays, including | |
| | all 4 bitewings and PAs of every | |
| | tooth. | |
| Dental | A CBCT system rotates around the | Voxel |
| cone-beam | patient and captures data using a | values |
| computed | cone-shaped x-ray beam. | |
| tomography | A CBCT is a 3D image. | |
| (CBCT) | | |
| Radiology | Free text radiology reports | Vector |
| reports | describing the findings and | representations |
| for dental | impression of dental or medical | of words |
| x-rays, | images | |
| CT scans, | | |
| or other | | |
| medical | | |
| images | | |
| Presence or | A list of dental findings of | Multi-hot |
| absence of | interest with the presence or | encoding |
| specific | absence of each one indicated | |
| dental | with a 1 or a 0, based on | |
| findings | natural language processing | |
| extracted | or named entity recognition | |
| from | applied to free text radiology | |
| radiology | reports for dental images. | |
| reports | Findings for x-ray images | |
| for dental | may include decay, | |
| images | caries/cavities, bone loss, | |
| | periapical infections, | |
| | radiolucencies, and | |
| | radioopacities. Findings | |
| | for CTs may include | |
| | multi-rooted teeth, | |
| | furcation involvement (bone | |
| | loss where roots bifurcate), | |
| | endodontic infections, | |
| | bone loss, distance between | |
| | teeth or implants and the | |
| | nerve, bone thickness such | |
| | as buccal bone thickness, | |
| | bone density, and infections. | |
| Intraoral | An intraoral scanner is a | 3D |
| scan | handheld device that | representation |
| | directly creates a digital | |
| | impression of the oral | |
| | cavity | |
| Dentlytec | The Dentlytec DigiProbe | Pixel values |
| DigiProbe | integrates 3D scanning and | for images, |
| output | imaging for teeth and | integers for |
| | automates periodontal charting. | pocket depth |
| | It includes photorealistic | |
| | images of teeth and gums, | |
| | automated pocket depth | |
| | measurement, and bleeding | |
| | identification. | |
| Perimetrics | The Perimetrics InnerView | Integer or |
| InnerView | system measures damping | floating- |
| measurements | characteristics of the | point number |
| | periodontium and associated | for each |
| | fixed structures including | measurement |
| | teeth and implants. It | |
| | provides data to quantify | |
| | tooth and dental implant | |
| | mobility. | |
| Bytesense | Measurements of tooth | Integer or |
| smart | grinding and sleep quality | floating- |
| nightguard | | point number |
| or other | | for each |
| nightguard | | measurement |
| sensors | | |
| Salivary | Oral DNA testing results | Sequence or |
| test of | can include screening | variant |
| oral DNA | that identifies bacterial | information |
| | risk for dental caries. | |
| Genetic | Genetic testing results | Sequence |
| testing or | can include genotyping of | or variant |
| “omics” | IL-6, SNP arrays, whole exome | information |
| | sequencing, or whole genome | |
| | sequencing. “Omics” can | |
| | include genomics, transcriptomics, | |
| | metabolomics, or proteomics. | |
| | Genetic testing or “omics” | |
| | may be applied to the patient | |
| | themselves and/or to their | |
| | microbiome. | |
| Physical | Description of a dentist's | Vector |
| examination | physical examination. The | representation |
| findings | physical examination may | of words, or |
| | include oral cancer screening | one-hot or |
| | based on visual inspection | multi-hot |
| | of the oral cavity, palpation | encoding of |
| | of lymph nodes, and a neck | categorical |
| | exam. | observations |
| Smoking | An indication of whether the | Integer: 0 |
| status | patient is currently smoking. | for nonsmoking, |
| | May also include whether | 1 for smoking. |
| | the patient has previously | Potentially |
| | smoked, and if so, how many | an integer or |
| | years ago they quit. | floating-point |
| | | number to |
| | | indicate |
| | | how long ago |
| | | the patient |
| | | quit smoking, |
| | | if relevant. |
| Demographics | Patient demographics such | Age as an |
| | as age, race, gender, or | integer Race |
| | ethnicity | as a one-hot |
| | | or multi-hot |
| | | vector Gender |
| | | as a one-hot |
| | | or multi-hot |
| | | vector Ethnicity |
| | | as a one-hot |
| | | or multi-hot |
| | | vector |
| Color | Color photos of the mouth including | Pixel values |
| photographs | photos of one or more teeth and/or | |
| of the | the patient's gums. Photos may | |
| mouth taken | provide visual information about | |
| during a | gum recession, tissue color, and | |
| physical | attached tissue. Photos may be | |
| examination | obtained using intraoral cameras | |
| | such as those manufactured by | |
| | MouthWatch. | |
| Color | Color photographs taken by | Pixel values |
| photos taken | dentists or assistants during a | |
| during a | procedure. | |
| procedure | | |
| Laboratory | Results of laboratory tests | Vector of |
| test | such as blood tests (e.g. CRP, | floating- |
| results | cytokines, BMP, CBC). | point numbers |
| | | or vector of |
| | | integers |
| | | (depending |
| | | on the |
| | | specific |
| | | lab tests |
| | | considered) |
| Existing | Existing dental diagnoses, which | Vector |
| dental | may be described as words, | representations |
| diagnoses | phrases, or codes within an | of words or |
| | ontology such as ICD. | multi-hot |
| | | encoding |
| | | (specifying |
| | | presence/absence |
| | | of multiple |
| | | diseases of |
| | | interest) |
| Dental | Past dental procedures, which | Vector |
| procedure | may be described as words, | representations |
| history | phrases, or codes within an | of words or |
| | ontology such as ADA (American | multi-hot |
| | DentalAssociation procedure | encoding |
| | codes). | (specifying |
| | | presence/absence |
| | | of dental |
| | | procedures of |
| | | interest) |
| Existing | Existing medical diagnoses such | Vector |
| medical | as heart disease, diabetes, or | representations |
| diagnoses | cancer, which may be described | of words or |
| | as words, phrases, or codes | multi-hot |
| | within an ontology such as | encoding |
| | SNOMED or ICD | (specifying |
| | | presence/absence |
| | | of multiple |
| | | diseases of |
| | | interest) |
| Probing | The probing depth as determined | Vector of |
| depth for | through a clinical exam of the | integers |
| all teeth | patient's mouth, for all | |
| | teeth | |
| Pain | Pain scores reported by the patient, | Each reported |
| scores | for example on a scale of 1 (no | pain score |
| | pain) to 10 (worst pain). Pain | is an integer |
| | scores may also be reported on | or |
| | a per-tooth basis. | floating-point |
| | | number |

Table 1 includes a non-exhaustive list of example data sources for providing textual, numerical, visual, and audible information, or any combination thereof, pertaining to the collection of patient data, dental data, patient's dental or systemic health, conditions, symptoms, or diagnoses. The data sources may include a patient's medical history or electronic health record (EHR), personal goals for dental function vs. aesthetics, patient information and concerns, comfort, and financial considerations. The data sources may be associated with a facility, clinic, hospital, laboratory, institution, machine or computing device, and information may be provided by a user, system, database, patient conversations or patient self-evaluation. In one or more embodiments, the multimodal artificial intelligence system may receive from the following data sources: full mouth x-rays (FMX), dental cone-beam computed tomography (CBCT), radiology reports for dental x-rays, CT scans, or other medical images, presence or absence of specific dental findings extracted from radiology reports for dental images, intraoral scan, Dentlytec DigiProbe output, Perimetrics InnerView measurements, Bytesense smart nightguard or other nightguard sensors, salivary test of oral DNA, genetic testing or “omics”, physical examination findings, smoking status, demographics, color photographs of the mouth taken during a physical examination, color photos taken during a procedure, laboratory test results, existing dental diagnoses, dental procedure history, existing medical diagnoses, probing depth for all teeth, and pain scores.

In certain embodiments, the raw data type or input format may include any of the following pixel values (e.g., 2D imaging), voxel values (e.g., 3D imaging), vector representations of words (e.g., radiology reports), multi-hot encoding (e.g., specific findings of interest from radiology reports), 3d representation (e.g., intraoral scan), pixel values and/or integers (e.g., pixel values for imaging, integers for direct measurements such as pocket depth), integer and/or floating-point numbers (e.g., pain score, duration the patient quit smoking), sequence or variant information, vector representation of words, or one-hot or multi-hot encoding of categorical observations (e.g., dental physical examination), integer values (e.g., age, smoking status: 0 for nonsmoking, 1 for smoking), one-hot and/or multi-hot vector (e.g., race, gender, ethnicity), vector of floating-point numbers and/or vector of integers (e.g., laboratory test results, depending on the specific lab tests considered), vector representations of words and/or multi-hot encoding (e.g., specifying presence/absence of multiple diseases of interest from an existing dental and/or medical diagnoses), vector representations of words or multi-hot encoding (e.g., specifying presence/absence of dental procedures of interest from an existing medical diagnoses), vector of integers (e.g., probing depth for all teeth).

In block 210, the plurality of data is preprocessed into a suitable format for AI/ML analysis. The data in Table 1 will be preprocessed according to standard techniques used for preparing data for AI systems. In certain embodiments, image data may be processed into a format suitable for AI analysis. DICOM images may be transformed into 2D or 3D arrays suitable for input into an AI system. For example, DICOMs may be transformed into 2D or 3D NumPy arrays. Pixel values may be centered and normalized. Image data augmentation techniques such as random flips and rotation or addition of Gaussian noise may be applied. Moreover, raw data may be screened to remove sensitive or confidential information. Moreover, raw data may be screened to remove sensitive or confidential information. As an example, the acquired raw data may be sent to a data handler for processing and sensitive data and/or tagged/labeled data may be removed.

In some embodiments, continuous variables may be centered and normalized using the mean and standard deviation of these variables in the training dataset. Further, categorical variables may be represented using one-hot or multi-hot encoding, and imputation of missing data may be performed using the mean of the training data for continuous variables, or the mode of the training data for categorical variables.

In block 215, the processed, screened, or formatted plurality of data (“hereinafter” processed data) is evaluated to prepare the plurality of processed data for AI/ML processing. For example, feature data from the processed data, if available, may be acquired and processed to determine one or more possible machine learning models. In some embodiments, feature data may be used to determine one or more statistical models and/or generate one or more expected features or data values. Feature data may be generated based on statistical models, screened data, other data, and observed or expected features. Feature vectors may be generated based on feature data. one or more generative models for processing feature vectors may be determined.

In one or more embodiments, one or more statistical models may be determined from the processed data and other patient information as provided herein. With reference to Table 2 below, the inputs and results from any or all phases of a treatment plan may be processed as raw patient and dental data that can be vital for understanding symptoms, outcomes, and subsequent procedures and treatment plans based on the patients present and past medical conditions. In embodiments, raw patient and dental data may be obtained and processed by the data management system 135 using a relevant a statistical model. The findings may then be used by the statistical model to determine a predictive function based on the data presented.

In block 220, one or more AI/ML models may be determined and configured for further processing the plurality of screened/formatted data. As described herein, the multimodal artificial intelligence system may determine a plurality of machine learning models prior to and during a treatment phase or procedure. In embodiments, the multimodal artificial intelligence system may leverage a neural network architecture. For example, a feedforward neural network may be used to process the non-image data described in Table 1, while a convolutional neural network or Vision Transformer architecture may be used to process the image data described in Table 1. A Transformer architecture may be applied to process any data represented as tokens. A unified architecture may be developed in which the following are concatenated: one or more representations produced by one or more feedforward neural networks, one or more representations produced by one or more convolutional neural networks, and/or one or more representations produced by one or more Transformers, and so forth. One or more additional neural network layers may then be applied to the concatenated representation in order to produce the final model output.

In block 225, the determined one or more AI/ML models may be configured for generating one or more treatment plans, procedures, and options prior to, during, or after one or more treatment phases as shown in FIG. 3 and described herein. As an example, a treatment planning model may be a multilabel classification model with outputs corresponding to different treatment options. If a patient has N teeth, and there are M different possible treatments that could be applied to each tooth, then the output may be a vector of length N×M specifying which treatment(s) should be applied to each tooth. Additionally, if there are P whole-mouth or systemic treatment options (e.g. antibiotics), the output vector may be of length (N×M)+P, corresponding to per-tooth treatments concatenated with systemic treatment options. In another example, the model may be a generative language model such as a generative pre-trained Transformer (GPT) model. In this embodiment the model generates text, and the generated text is the treatment plan represented in natural language. In another example, the system may generate multiple different treatment options, with or without further outputs specifying the cost, treatment duration, or aesthetic vs. functional focus of one or more of the generated treatment options.

In many embodiments, a treatment planning model may be developed to predict dental treatment plan(s) for a variety of different dental conditions including gingivitis, cavities, dentin hypersensitivity, halitosis, impacted teeth, chipped teeth, broken teeth, crooked teeth, stained teeth, tooth pain, malocclusion, tooth erosion, mouth ulcers, bruxism, and/or temporomandibular joint disorders. Further, the treatment planning model may be used during a procedure whereby a dentist or other care team member may enter new dental or patient data inputs or parameters. For example, a dentist or other care team may take a photograph of a patient during a procedure and provide the photograph as an input to the treatment planning model in order to receive feedback about the next steps needed in the patient's care. This use case may involve the AI treatment planning model helping to resolve unexpected issues that arise during a procedure.

In some embodiments, a plurality of AI/ML models may be executed to create a unified treatment plan for an individual patient that addresses one or more dental conditions. Further, the treatment planning model may be configured to determine a treatment plan that includes multiple alternative treatment plan options, which may vary in complexity, cost, or likely outcome. Each treatment plan option may describe different treatments, interventions, or actions in chronological order.

The inputs to the model may include any or all of the patient data shown in Table 1. Additionally, the model may also include a description of the patient's personal treatment goals. The patient's personal treatment goals may be described in natural language and represented using vector representations of words. The treatment goals may be written down by the patient or spoken by the patient and then transcribed using manual human effort or an AI speech-to-text system. The patient's personal treatment goals may specify one or more patient desires, such as restoring or improving oral function, oral health, or systemic health, improving aesthetics, maintaining low treatment cost, and/or alleviating pain. In another example, the patient's personal treatment goals may be represented as the output of a survey question that asks the patient to select which of a certain set of treatment considerations are most important to them-such as aesthetics, affordability, comfort, or functionality. In this case the patient's treatment goals may be represented as a one-hot or multi-hot vector.

Each treatment plan option may include one or more treatment plan phases. The proposed machine learning models may be performed during any phase list below in Table 2 and described in FIG. 3. The dentist, care team member, or user may execute and/or update inputs for a treatment plan model in-situ during or after each procedure.

Examples of possible phases are shown in Table 2. Not all phases are required. Any phase title may occur one or more times within a treatment plan option. Phases may include descriptions of specific interventions on particular teeth. A treatment plan option may be comprehensive, or a “band aid” approach solving only one specific problem. An example treatment plan is described in FIG. 3. The list of treatment plan phases in Table 2 is a non-exhaustive list of phases. The treatment plan phases listed below may occur during a patient treatment plan, visit or procedure.

TABLE 2
Phases of a Treatment Plan Option.

| Phase | Description |
|---|---|
| Initial | The first phase, which may include immediate treatment |
| Phase | needs. |
| Periodontal | A phase that addresses the structures surrounding and |
| Phase | supporting the teeth, such as the gums. |
| Provisional | In a provisional phase, patients may be given temporary |
| Phase | replacement of missing teeth via removable, tooth |
| | supported, or implant supported temporary solutions. |
| Surgical | During a surgical phase, the patient may undergo surgical |
| Phase | operations. |
| Endodontic | An endodontic phase involves the soft tissues inside a tooth |
| Phase | (the dental pulp). |
| Diagnostic | A diagnostic phase involves the patient undergoing one or |
| Phase | more diagnostic tests to better understand the etiology of |
| | their oral health issues. |
| Restorative | This phase involves fixes to a patient's teeth such a |
| Phase | realignment of crooked teeth or treatment of gaps with |
| | implants. |
| Maintenance | In this phase the patient's dental health is monitored |
| Phase | and maintained. |
| Referral | A patient may be sent to a specialist for a referral or |
| Phase | consultation. A patient may be referred for imaging, e.g. |
| | to obtain a CT scan. |
| Imaging | In this phase the patient may be imaged using x-ray or CT |
| Phase | technology. |
| Laboratory | In this phase the patient may undergo one or more |
| Phase | laboratory tests, such as a blood test. The patient may |
| | also interact with a laboratory technician or other |
| | laboratory personnel, for example to check fit of a |
| | manufactured item. |

In many embodiments, the dataset for training and evaluation may be collected from electronic dental record software. For a multilabel classification model, any free text descriptions of treatment plans may be transformed into the proposed (N×M)+P output vector previously described using manual human labor or using a natural language processing framework that automatically extracts structured data from free text. For a generative language model, free text treatment plan descriptions already present in the electronic medical record may be used directly during the training process. The input data will be collected at a certain time point, and the labels (outputs) will be collected at a future time point—for example, 5 years into the future or 10 years into the future. The final trained model will therefore be able to predict a patient's future risk of disease.

As an example, a dental foundation model may be trained to learn the data distribution of patient dental data. The data used for training may any or all of the data sources shown in Table 1, as well as data scraped from the Internet, including dental research papers, publicly available genetic datasets, publicly available dental or medical imaging datasets, or articles related to dentistry. This model may be a generative model implemented as a neural network, for example using a Transformer architecture. The data may be represented as tokens and the training objective may be a next token prediction objective.

In block 230, executing one or more prediction models to predict patient's dental or systemic diseases, symptoms, conditions, outcomes, success, and referrals (e.g., subsequent procedures and treatments) based on the patients present and past medical conditions. In embodiments, one or more AI/ML models may be determined and configured for predicting a patient's future risk of dental or systemic disease. In some embodiments, a multilabel classification model may be developed to predict a patient's future risk of oral or systemic disease based on dental health information and/or general health information. The model may also forecast long-term oral health outcomes based on current data. As an example, the inputs to the model may include any or all patient data elements shown in Table 1. In order to predict future risk of disease, the model used may be a multilabel classification model. For example, the output layer of the neural network may have a sigmoid function applied to each output neuron, so that the model is suitable for multilabel classification, i.e., a task in which more than one output may be predicted simultaneously. This can be useful as the patient may be at risk of developing multiple diseases in the future. Output dental diseases, conditions, or symptoms that may be predicted include, for example, periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and/or progression of cavities (incipient vs. in dentin, duration of time for cavity to progress into the nerve/pulp). Systemic diseases, conditions, or symptoms that may be predicted include diabetes, cancers, heart attacks/cardiovascular disease, stroke, inflammatory disease, and arthritis.

In embodiments, one or more AI/ML models may be determined and configured as a treatment success model for predicting treatment success by comparing pre-treatment patient data with post-treatment patient data, or by analyzing post-treatment patient data alone. As an example, a comparison model may be used. The model inputs may include any or all of the data shown in Table 1, at a pre-treatment time point, plus any or all of the data shown in Table 1 at a post-treatment time point. In a post-treatment data model, only the post-treatment time point data will be included as input. The model may be implemented as a multiclass classification model with three outputs: treatment success, treatment survival, or treatment failure. The model may also be implemented using a regression objective to predict a numerical score indicative of the degree of treatment success.

In some embodiments, the dataset for training and evaluation may be collected from electronic health record software. For a comparison model, pre-treatment and post-treatment data will be obtained—for example, x-rays before treatment along with x-rays after treatment. The labels indicating treatment success, survival, or failure may be automatically extracted from dental notes or other dental records using natural language processing. The labels may also be manually acquired using human effort, for example by recruiting dentists to participate in a labeling process. Multiple dentists may label the same record and a consensus label may be obtained.

In embodiments, one or more AI/ML models may be determined and configured as a referral model for predicting whether a patient needs a referral and if so to what specialist. This model may be used to assist a dentist and/or hygienist to know when a patient needs referral. For example, in a situation where a patient has returned repeatedly for follow up and their oral health is not improving sufficiently, the model may be able to identify that the patient requires referral to a periodontist.

In block 235, displaying, via a graphical user interface (GUI), the inputs and/or output(s) generated by one or more AI/ML models. In some embodiments, the MAI system GUI may prompt a user, dentist, or care staff member for updated inputs or additional information at any point prior to, during, or after a treatment plan phase. Moreover, the multimodal artificial intelligence system may be configured or required to prompt a user for entry of inputs or additional information during at any point prior to, during, or after a treatment plan phase. The MAI system GUI may then display all outputs/findings, treatment plans, procedures, predictions, referrals, inputs, inputs, and so forth. In embodiments, the MAI system GUI may present and print-out the inputs and outputs as an organized list whereby each treatment plan phase serves as a header and all inputs and outputs for the corresponding phase are grouped together.

### Treatment Plan Phases for an Example Dental Treatment Plan

### Treatment Example 1

FIG. 3 illustrates embodiments of a dental treatment plan that may be implemented by the MAI system 100 of FIG. 1. As listed in Table 2, a patient treatment plan may include several plan phases for addressing, monitoring, and maintaining a patient's oral and systemic health and various concerns. Blocks shown in FIG. 3 may represent one or more processes, methods or subroutines, carried out in the exemplary treatment plan method of FIG. 2. Further, FIGS. 1, 2, and 4 show example embodiments of carrying out the method of FIG. 3 for acquiring, preparing, and processing a plurality of patient data using a multimodal artificial intelligence system to generate a unified perspective of a dental patient's diagnosis that includes dental disease risk prediction and treatment planning.

With reference to FIG. 3 and Table 1, the following example of treatment plan phases includes example inputs obtained from the patient for processing by the MAI system described herein. In block 305, an example patient treatment plan may include inputs as follows. In the initial phase, a patient may provide consent to allow data to be collected through, for example, clinical photography, Alginate Impressions with diagnostic casts, Facebow transfer record, CR bite record and articulator mounting of casts, and the like. The initial phase may include addressing immediate treatment needs.

In block 310, an example patient treatment plan may include a periodontal phase for addressing the structures surrounding and supporting the teeth, such as the gums. In the periodontal phase, a patient may be given oral hygiene instructions, provided with scaling and root planning, and given a 4-6-week periodontal re-evaluation schedule, and the like.

In block 315, an example patient treatment plan may include a provisional phase that involves giving patients temporary replacement of missing teeth via removable, tooth supported, or implant supported temporary solutions. In the provisional phase, a patient may undergo removal of existing porcelain fused to metal (PFM) crowns from teeth numbers 4, 10p-11, and 13. Evaluation of restorability of each tooth, and placement of provisional restorations. Further, a subsequent provisional phase may include removal of existing fixed partial dentures (FPD) from teeth numbers 18-20, restoration of tooth number 21 denture with composite to restore denture contour and restore bridge with composite (or amalgam/gold inlay), and placement of a provisional restoration for FPD teeth numbers 18-20. Moreover, a subsequent provisional phase may include delivery of maxillary treatment for removable partial denture (RPD) to replace teeth numbers 5, 9 and 12 for esthetics, and determination that no prosthesis is in mandible during healing phase. Further, a subsequent provisional phase may include preparation of teeth numbers 6, 7, and 8 for porcelain veneer restorations based on diagnostic wax-up, and placement of provisional restorations. Moreover, a subsequent provisional phase may include placement of provisional restorations for teeth numbers 3i, 5i, 9i-10p, 12i, 14i, 19i, and 29i-30i.

In block 320, an example patient treatment plan may include a surgical phase where the patient may undergo surgical operations. In the surgical phase, a patient treatment may include section provisional restoration distal to tooth number 20, and extraction of teeth numbers: 2-3, 5, 9, 12, 14-15, 18, 29, and 31-32. Further, a subsequent surgical phase may include fabrication of surgical guide and implant placement for teeth numbers 3, 5, 9, 12 and 14. Moreover, a subsequent surgical phase may include fabrication of surgical guide and implant placement for teeth numbers 19, 29, and 30. Further, a subsequent surgical phase may include a second stage surgery to uncover implants and placement of healing abutments.

In block 325, an example patient treatment plan may include an endodontic phase that involves treatments for the soft tissues inside a tooth (i.e., the dental pulp). In the endodontic phase, a patient may undergo a root canal treatment (RCT) of tooth number 4.

In block 330, an example patient treatment plan may include a diagnostic phase that involves the patient undergoing one or more diagnostic tests to better understand the etiology of their oral health issues. The diagnostic phase may include a complete diagnostic wax-up, fabrication of radiographic guides, CT scan of maxilla and mandible, and evaluation of bone quantity and quality for possible sinus lifts or bone augmentation.

In block 335, an example patient treatment plan may include a restorative phase that involves fixes to a patient's teeth such a realignment of crooked teeth or treatment of gaps with implants. In the restorative phase, a patient treatment may include delivering definitive restorations for teeth numbers 3i, 4 PFM, 5i, 6 Veneer, 7 Veneer, 8 Veneer, 9i-10p, 11 PFM, 12i, 13 PFM, 14i, 19i, 20 PFM, and 29i-30i, and delivering occlusal night guard.

In block 340, an example patient treatment plan may include a maintenance phase that involves monitoring and maintaining the patient's dental health. In the maintenance phase, a 6-month prosthodontic recall and 3-6-month periodontal recall may be performed.

In block 345, an example patient treatment plan may include a referral phase whereby the patient may receive one or more referrals for a specialist or imaging based on the findings of the multimodal artificial intelligence system.

In block 350, an example patient treatment plan may include an imaging phase whereby the patient may be imaged using x-ray or CT technology. As described above, the patient may be referred for imaging (e.g., to obtain a CT scan) the imagery results may be entered manually or remotely into the MAI system.

In block 355, an example patient treatment plan may include a laboratory phase whereby the patient may undergo one or more laboratory tests, such as a blood test. The patient may also interact with a laboratory technician or other laboratory personnel, for example to check the fit of a manufactured item. The laboratory results may be entered manually or remotely into the MAI system.

### Treatment Example 2

In block 305, in an initial phase, a patient may provide consent to allow data to be collected through, for example, clinical photography, Alginate Impressions with diagnostic casts, Facebow transfer record, CR bite record and articulator mounting of casts, and the like. The initial phase may include addressing immediate treatment needs.

In block 310, in a periodontal phase, a patient may be given oral hygiene instructions, provided with scaling and root planning, and given a 4-6-week periodontal re-evaluation schedule, and the like.

In block 315, in a provisional phase, a patient may undergo removal of existing cantilever FPD for teeth numbers 10p-11, and preparation of teeth numbers 2, 6, 15 with similar path of insertion in preparation of a full arch fixed provisional restoration, and preparation of individual provisional restorations for teeth numbers 2, 6, 10p-11 and 15. Further, a subsequent provisional phase may include removal of existing fixed partial dentures (FPD) from teeth numbers 18-20, restoration of tooth number 21 denture with composite to restore denture contour and restore bridge with composite (or amalgam/gold inlay), and placement of a provisional restoration for FPD teeth numbers 18-20. Moreover,

With reference to block 330, in a diagnostic phase, the treatment process includes a complete diagnostic wax-up.

In block 320, in a surgical phase, a patient treatment may include section provisional restoration distal to tooth number 20, and extraction of teeth numbers: 3-5, 7-9, 12-15, 18, 29, and 31.

With reference to block 315, in a subsequent provisional phase may include delivery of maxillary FPD provisional restoration for teeth numbers 2-3p-4p-5p-6-7p-8p-9p-10p-11-12p-13p-14p-15, and determination that no prosthesis is in mandible during healing phase.

With reference to block 330, in a subsequent diagnostic phase, the treatment process includes fabrication of maxillary radiographic guide indexed to remaining prepared teeth, fabrication of mandibular radiographic guide indexed to remaining dentition, CT scan of maxilla and mandible, and evaluation of bone quantity and quality for possible sinus lifts or bone augmentation.

With reference to block 320, in a subsequent surgical phase, a patient treatment may include fabrication of surgical guide, implant placement for teeth numbers 3, 4, 5, 7, 10, and 12-14, and relieving provisional restoration over implant sites. Further, a subsequent surgical phase may include fabrication of surgical guide and implant placement for teeth numbers 19, 29, and 30. Moreover, a subsequent surgical phase may include a second stage surgery to uncover implants, placement of healing abutments, and extraction of tooth number 32 (maintained as vertical stop).

With reference to block 315, in a subsequent provisional phase, a patient treatment may include placement of max provisional restorations for teeth numbers 3i-4i-5i-6p-7i-8p-9p-10i-11p-12i-13i-14i, and placement of mand provisional restorations for teeth numbers 19i, 29i-30i.

In block 335, in a restorative phase, a patient treatment may include delivery of definitive restorations for teeth numbers 3i-4i-5i-6p, 7i-8p-9p-10i, 11p-12i-13i-14i, 19i, 20 PFM, 29i-30i, and delivery of occlusal night guard.

In block 340, in a maintenance phase, a patient treatment may include a 6-month prosthodontic recall and a 3-6-month periodontal recall.

### Definitions

A “patient data”, “dental data”, “collected patient data”, “raw patient data”, “raw dental data”, or “relevant dental data” as used herein includes, but is not limited to, data of a patient that can be interpreted, collected, processed, presented, and displayed to at least one of an interface, machine (e.g., robotic systems), or computing device integrated with the multimodal AI-driven system that can be used for assessing and/or addressing at least a health, comfort, dental aesthetic, or appearance of the patient. The data of a patient may include dental data, patient data, and any other data acquired for the patient.

A “similar”, “same”, “related”, or “relevant” as used herein in regard to patient health, dental data, patient data, patient treatment or diagnosis, includes, but is not limited to, comparable data from one or more patients or subjects having at least one symptom, condition, or disease in common with the patient being evaluated and/or diagnosed through one or more ML/AI models of the present disclosure.

A “processed dental data”, or “processed data”, as used herein includes, but is not limited to, data of a patient that is processed, formatted, adjusted, edited, or otherwise manipulated for training a machine learning model and/or processing by a multimodal AI-driven system, method, or logic.

### Computing Device Embodiment

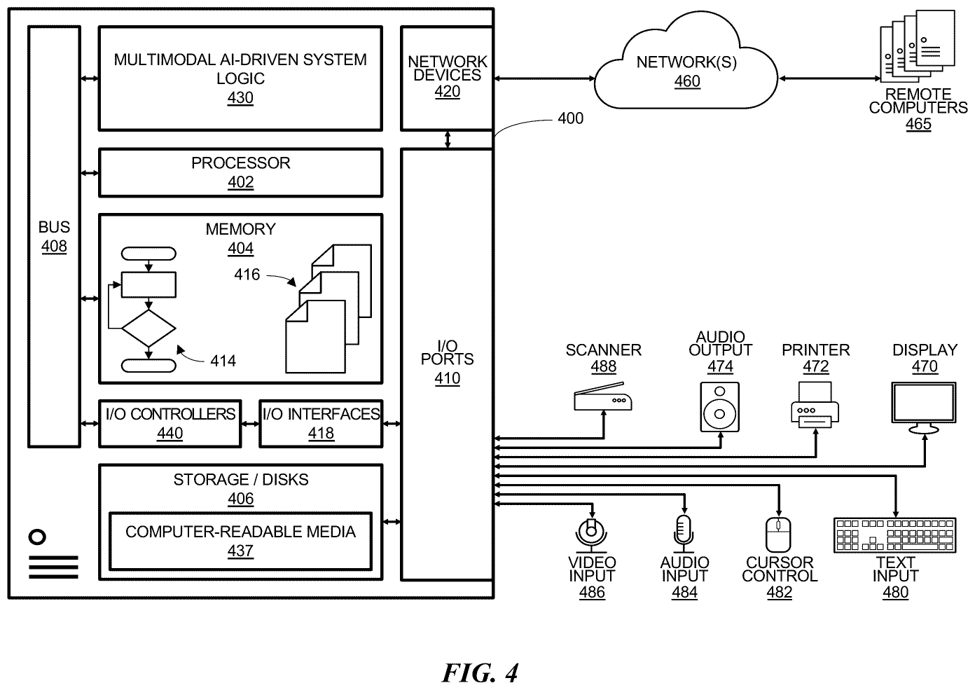

FIG. 4 illustrates an example computing device that is configured and/or programmed as a special purpose computing device with one or more of the example systems and methods described herein, and/or equivalents. The example computing device may be a computer 400 that includes at least one hardware processor 402, a memory 404, and input/output ports 410 operably connected by a bus 408. In one example, the computer 400 may include multimodal AI-driven system logic 430 configured to generate a unified perspective of a dental patient's diagnosis using multimodal artificial intelligence to facilitate dental disease risk predictions, prediction of a patient's future risk of oral or systemic disease, a dental treatment plan, an individualized patient treatment plan, and an assessment of the success of a treatment and/or procedure as the MAI system 100 (FIG. 1 and associated figures). The multimodal AI-driven system logic 430 creates a unified perspective of a dental patient's diagnosis for patients using a plurality of dental data from the patient processed by a plurality of AI/ML models (multimodal AI) to facilitate dental disease risk prediction and dental treatment planning.

The multimodal AI-driven system logic 430 can process a plurality of dental data to generate a unified perspective of a dental patient's diagnosis, that can include, predicting treatment success based on the patient's dental data, predicting a patient's future risk of dental or systemic disease, predicting whether or not a patient requires referral to a specialist, facilitating a patient's personal treatment goal(s), providing a dental treatment plan that includes multiple alternative treatment plan options based on complexity, cost, or likely outcome of a procedure, assessing the success of a generated treatment by acquiring a post-treatment patient data, as well as a variety of dental treatment plans and treatment options. In different examples, the logic 430 may be implemented in hardware, a non-transitory computer-readable medium 437 with stored instructions, firmware, and/or combinations thereof. While the logic 430 is illustrated as a hardware component attached to the bus 408, it is to be appreciated that in other embodiments, the logic 430 could be implemented in the processor 402, stored in memory 404, or stored in disk 406.

In embodiments, logic 430 or the computer is a means (e.g., structure: hardware, non-transitory computer-readable medium, firmware) for performing the actions described. In some embodiments, the computing device may be a server operating in a cloud computing system, a server configured in a Software as a Service (SaaS) architecture, a smart phone, laptop, tablet computing device, and so on.

The means may be implemented, for example, as an ASIC programmed to facilitate serial or parallel execution of generative, neural network, multiclass classification, multilabel classification, generative pre-trained Transformer, or dental foundation models, or other AI/ML models through a graphical user interface creating a unified perspective of a dental patient's diagnosis using a plurality of dental data and a plurality of AI/ML models (multimodal AI) to facilitate dental or systemic disease risk prediction, predicting treatment success, dental treatment planning, and personal treatment goals and plans. The means may also be implemented as stored computer executable instructions that are presented to computer 400 as data 416, which may be in the form of a data structure or data structures, that are temporarily stored in memory 404 and then executed by processor 402.

Logic 430 may also provide means (e.g., hardware, non-transitory computer-readable medium that stores executable instructions, firmware) for performing one or more of the disclosed functions and/or combinations of the functions.

Generally describing an example configuration of the computer 400, the processor 402 may be a variety of various processors including dual microprocessor and other multi-processor architectures. A memory 404 may include volatile memory and/or non-volatile memory. Non-volatile memory may include, for example, ROM, PROM, and so on. Volatile memory may include, for example, RAM, SRAM, DRAM, and so on.

A storage disk 406 may be operably connected to the computer 400 via, for example, an input/output (I/O) interface (e.g., card, device) 418 and an input/output port 410 that are controlled by at least an input/output (I/O) controller 440. The disk 406 may be, for example, a magnetic disk drive, a solid-state disk drive, a floppy disk drive, a tape drive, a Zip drive, a flash memory card, a memory stick, and so on. Furthermore, the disk 406 may be a CD-ROM drive, a CD-R drive, a CD-RW drive, a DVD ROM, and so on. The memory 404 can store a process 414 and/or a data 416, which may be in the form of a data structure, for example. The disk 406 and/or the memory 404 can store an operating system that controls and allocates resources of the computer 400.

The computer 400 may interact with, control, and/or be controlled by input/output (I/O) devices via the input/output (I/O) controller 440, the I/O interfaces 418, and the input/output ports 410. Input/output devices may include, for example, one or more displays 470, printers 472 (such as inkjet, laser, or 3D printers), audio output devices 474 (such as speakers or headphones), text input devices 480 (such as keyboards), cursor control devices 482 for pointing and selection inputs (such as mice, trackballs, touch screens, joysticks, pointing sticks, electronic styluses, electronic pen tablets), audio input devices 484 (such as microphones, synthesizers or external audio players), video input devices 486 (such as video and still cameras, or external video players), image scanners 488, video cards (not shown), disks 406, network devices 420, and so on. The input/output ports 410 may include, for example, serial ports, parallel ports, and USB ports.

The computer 400 can operate in a network environment and thus may be connected to the network devices 420 via the I/O interfaces 418, and/or the I/O ports 410. Through the network devices 420, the computer 400 may interact with a network 460. Through the network, the computer 400 may be logically connected to remote computers 465. Networks with which the computer 400 may interact include, but are not limited to, a LAN, a WAN, and other networks.

### Definitions and Other Embodiments

In another embodiment, the described methods and/or their equivalents may be implemented with computer executable instructions. Thus, in embodiments, a non-transitory computer readable/storage medium is configured with stored computer executable instructions of an algorithm/executable application that when executed by a machine(s) cause the machine(s) (and/or associated components) to perform the method. Example machines include but are not limited to a processor, a computer, a server operating in a cloud computing system, a server configured in a Software as a Service (SaaS) architecture, a smart phone, and so on. In embodiments, a computing device is implemented with one or more executable algorithms that are configured to perform any of the disclosed methods.

In one or more embodiments, the disclosed methods or their equivalents are performed by either: computer hardware configured to perform the method; or computer instructions embodied in a module stored in a non-transitory computer-readable medium where the instructions are configured as an executable algorithm configured to perform the method when executed by at least a processor of a computing device.

While for purposes of simplicity of explanation, the illustrated methodologies in the figures are shown and described as a series of blocks of an algorithm, it is to be appreciated that the methodologies are not limited by the order of the blocks. Some blocks can occur in different orders and/or concurrently with other blocks from that shown and described. Moreover, less than all the illustrated blocks may be used to implement an example methodology. Blocks may be combined or separated into multiple actions/components. Furthermore, additional and/or alternative methodologies can employ additional actions that are not illustrated in blocks. The methods described herein are limited to statutory subject matter under 35 U.S.C. § 101.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “embodiments”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in embodiments” does not necessarily refer to the same embodiment, though it may.

A “data structure”, as used herein, is an organization of data in a computing system that is stored in a memory, a storage device, or other computerized system. A data structure may be any one of, for example, a data field, a data file, a data array, a data record, a database, a data table, a graph, a tree, a linked list, and so on. A data structure may be formed from and contain many other data structures (e.g., a database includes many data records). Other examples of data structures are possible as well, in accordance with other embodiments.

“Computer-readable medium” or “computer storage medium”, as used herein, refers to a non-transitory medium that stores instructions and/or data configured to perform one or more of the disclosed functions when executed. Data may function as instructions in some embodiments. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an application specific integrated circuit (ASIC), a programmable logic device, a compact disk (CD), other optical medium, a random access memory (RAM), a read only memory (ROM), a memory chip or card, a memory stick, solid state storage device (SSD), flash drive, and other media from which a computer, a processor or other electronic device can function. Each type of media, if selected for implementation in embodiments, may include stored instructions of an algorithm configured to perform one or more of the disclosed and/or claimed functions. Computer-readable media described herein are limited to statutory subject matter under 35 U.S.C. § 101.

“Logic”, as used herein, represents a component that is implemented with computer or electrical hardware, a non-transitory medium with stored instructions of an executable application or program module, and/or combinations of these to perform any of the functions or actions as disclosed herein, and/or to cause a function or action from another logic, method, and/or system to be performed as disclosed herein. Equivalent logic may include firmware, a microprocessor programmed with an algorithm, a discrete logic (e.g., ASIC), at least one circuit, an analog circuit, a digital circuit, a programmed logic device, a memory device containing instructions of an algorithm, and so on, any of which may be configured to perform one or more of the disclosed functions. In embodiments, logic may include one or more gates, combinations of gates, or other circuit components configured to perform one or more of the disclosed functions. Where multiple logics are described, it may be possible to incorporate the multiple logics into one logic. Similarly, where a single logic is described, it may be possible to distribute that single logic between multiple logics. In embodiments, one or more of these logics are corresponding structure associated with performing the disclosed and/or claimed functions. Choice of which type of logic to implement may be based on desired system conditions or specifications. For example, if greater speed is a consideration, then hardware would be selected to implement the functions. If a lower cost is a consideration, then stored instructions/executable applications would be selected to implement the functions. Logic is limited to statutory subject matter under 35 U.S.C. § 101.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, non-transitory computer-readable medium). Logical and/or physical communication channels can be used to create an operable connection.

“User”, as used herein, includes but is not limited to one or more persons, computers or other devices, machines (e.g., robotic system or robot), or combinations of these.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

What is claimed is:

1. A computer-implemented method comprising:
acquiring a plurality of dental data for a patient;
processing the plurality of dental data for consumption by a machine learning model;
configuring the machine learning model to be a treatment planning machine learning model;
generating, via the treatment planning machine learning model, a diagnosis of one or more conditions and a patient treatment plan that includes at least one of a dental treatment plan and a predicted dental treatment plan; and
displaying, via a user interface, the generated patient treatment plan.

2. The method of claim 1, wherein the user interface comprises a graphical user interface (GUI).

3. The method of claim 1, further comprising predicting, using the treatment planning machine learning model, a dental health score, and a gum health score of the patient.

4. The method of claim 1, further comprising predicting, using the treatment planning machine learning model, a progression of an existing dental disease.

5. The method of claim 1, wherein the plurality of dental data includes at least one of: dental images, dental radiology reports, genetic testing results, physical examination findings, smoking status, demographics, color photographs, laboratory tests, existing diagnoses, dental procedure history, gum disease metrics, probing depth, and pain scores.

6. The method of claim 1, further comprising training the treatment planning machine learning model on an online resource, online database, or collection of patient dental data from an electronic dental record.

7. The method of claim 1, further comprising predicting, using the treatment planning machine learning model, a priority level for one or more elements of the generated patient treatment plan.

8. The method of claim 7, further comprising assessing the success of the generated patient treatment plan by acquiring a post-treatment plurality of patient data, providing the post-treatment patient data to a progression machine learning model along with the pre-treatment patient data, and receiving a model output indicating treatment success.

9. The method of claim 1, further comprising configuring the dental treatment plan to include a unified treatment plan for an individual patient, the unified treatment plan addresses one or more of the following dental conditions: gingivitis, tongue disease, cavities, dentin hypersensitivity, halitosis, impacted teeth, chipped teeth, broken teeth, crooked teeth, stained teeth, tooth pain, malocclusion, tooth erosion, mouth ulcers, bruxism, and temporomandibular joint disorders.

10. The method of claim 1, further comprising configuring the dental treatment plan to include a plurality of alternative treatment plan options based on varying complexity, cost, or likely outcome of a treatment plan, and wherein each of the plurality of alternative treatment plan options describes different treatments, interventions, or actions in chronological order.

11. The method of claim 1, further comprising configuring the dental treatment plan to include one or more of the following phases: initial phase, periodontal phase, provisional phase, surgical phase, endodontic phase, diagnostic phase, restorative phase, maintenance phase, referral phase, imaging phase, and laboratory phase.

12. The method of claim 1, further comprising configuring the treatment planning machine learning model to be one of a neural network, a multilabel classification model, a generative pre-trained Transformer, or a dental foundation model.

13. The method of claim 1, further comprising using the treatment planning machine learning model during a procedure to receive feedback on the next steps in the patient's immediate care.

14. The method of claim 1, wherein the plurality of dental data includes the patient's personal treatment goals represented in a natural language, a vector representation of words or text, a one-hot vector, or a multi-hot vector, and wherein the personal treatment goals include at least one of the following concepts: restoring or improving oral function, oral health, or systemic health, improving aesthetics, maintaining low treatment cost, alleviating pain, and increasing comfort.

15. The method of claim 1, further comprising integrating a robotics system with the treatment planning machine learning model, wherein the robotics system carries out one or more of the proposed treatments via a direct physical intervention upon the patient.

16. The method of claim 1, further comprising predicting, using the treatment planning machine learning model, whether or not a patient requires a referral to a specialist and if so, what specialist the patient should see.

17. The method of claim 1, further comprising predicting, using the treatment success machine learning model, a treatment success by comparing a pre-treatment patient data with a post-treatment patient data, or by analyzing the post-treatment patient data alone.

18. The method of claim 1, further comprising predicting, using the machine learning model, tongue health, one or more specific tongue conditions, or one or more systemic conditions that can manifest through abnormal tongue appearance.

19. A computer-implemented method comprising:
acquiring a plurality of dental data for a patient;
processing the plurality of dental data for consumption by a machine learning model;
predicting, using a multilabel classification machine learning model, a patient's future risk of dental or systemic disease based on at least one of the processed plurality of dental data; and
displaying, via a user interface, the multilabel classification machine learning model predictions of future disease risk, wherein the predicted future disease risk includes at least one of the following predicted diseases: periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and progression of cavities (incipient vs. in dentin, duration of time for cavity to progress into the nerve/pulp), diabetes, cancers, heart attacks/cardiovascular disease, stroke, inflammatory disease, and/or arthritis.

20. A computer-implemented method comprising:
acquiring a plurality of dental data for a patient;
processing the plurality of dental data for consumption by a machine learning model;
predicting, using a multiclass classification machine learning model, a patient's treatment success based on at least one of the processed plurality of dental data, wherein an output classes of the multiclass classification machine learning model includes at least one of a treatment success, a treatment survival, and a treatment failure; and
displaying, via a user interface, the multiclass classification machine learning model predictions.

21. A system comprising:
a computing device configured to:
acquire a plurality of dental data for a patient;
process the plurality of dental data for consumption by a machine learning model;
configure the machine learning model to be a treatment planning machine learning model;
generate, via the treatment planning machine learning model, a patient treatment plan that includes at least one of a dental treatment plan and a predicted dental treatment plan; and
a user interface configured to display the generated patient treatment plan.

22. A system comprising:
a computing device configured to:
acquire a plurality of dental data for a patient;
process the plurality of dental data for consumption by a machine learning model;
predict, using a multilabel classification machine learning model, a patient's future risk of dental or systemic disease based on at least one of the processed plurality of dental data; and
user a user interface to displaying the multilabel classification machine learning model predictions of future disease risk, wherein the predicted future disease risk includes at least one of the following predicted diseases: periodontal disease, tongue diseases, diseases that manifest through abnormal tongue appearance such as vitamin deficiency, bone loss, amount of tooth mobility, bone breakdown, rate of bone breakdown, cracked teeth, broken teeth, cavities, and progression of cavities (incipient vs. in dentin, duration of time for cavity to progress into the nerve/pulp), diabetes, cancers, heart attacks/cardiovascular disease, stroke, inflammatory disease, and/or arthritis.

23. A system comprising:
a computing device configured to:
acquire a plurality of dental data for a patient;
process the plurality of dental data for consumption by a machine learning model;
predict, using a multiclass classification machine learning model, a patient's treatment success based on at least one of the processed plurality of dental data, wherein an output classes of the multiclass classification machine learning model includes at least one of a treatment success, a treatment survival, and a treatment failure; and
use a user interface to display the multiclass classification machine learning model predictions.
